(19) United States
(12) Patent Application Publication
(10) Pub. No.: US 2007/0141147 A1
(43) Pub. Date: Jun. 21, 2007

(54) SEQUENTIAL RELEASE PHARMACEUTICAL FORMULATIONS

(75) Inventors: Matthew F. Heil, Duluth, GA (US); Glynn Wilson, Duluth, GA (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Auriga Laboratories, Inc.

(21) Appl. No.: 11/461,238

(22) Filed: Jul. 31, 2006

Related U.S. Application Data
(60) Provisional application No. 60/752,057, filed on Dec. 21, 2005.
(60) Provisional application No. 60/761,766, filed on Jan. 25, 2006.
(60) Provisional application No. 60/791,408, filed on Apr. 13, 2006.

(51) Int. Cl. A61K 31/55; A61K 31/485; A61K 9/22; A61K 31/137; A01N 31/08

(52) U.S. Cl. 424/468; 514/282; 514/304; 514/649; 514/217.05; 514/731

(57) ABSTRACT

A mixed-release tablet or capsule formulation including vehicles for the delivery of a plurality of drugs in various combinations of immediate release, extended release, and/or delayed release modes over a predetermined time period have been developed, which provide for controlled release not just of the drugs, but controlled release that is designed to create more effective coordination between the drugs being delivered. The drugs can be any medically and/or physiologically appropriate combination of drugs and active ingredients, preferably decongestant drugs, antihistamines, expectorants, antitussives, cough suppressants, and drying agents.

### CROSS-REFERENCE TO RELATED APPLICATIONS

Priority is claimed to U.S. application Nos. 60/752,057, filed Dec. 21, 2005; 60/761,766 filed Jan. 25, 2006; and 60/791,408 filed Apr. 13, 2006

### FIELD OF THE INVENTION

The present invention provides a mixed-release formulation that contains a plurality of active therapeutic agents and provides continuous immediate and/or extended dosing and delayed dosing for a predetermined time period, up to at least 12 hours.

### BACKGROUND OF THE INVENTION

Existing methods for the treatment of a variety of conditions involve the administration of more than one active agent using formulations that allow for immediate as well as controlled release. The drug delivery profiles of such formulations are simple, usually involving the immediate release of an active ingredient, followed by the sustained release of the same ingredient, or a different active ingredient, delayed for a certain period of time. However, many of these formulations fail to take advantage of combining multiple controlled, delayed, or immediate release profiles within a single preparation other than just combinations of immediate and delayed release. In particular, present methods for the treatment of cold or allergic rhinitis symptoms rely upon a wide variety of agents, most principally antihistamines (e.g., Chlorpheniramine), expectorants e.g., guaifenesin), decongestants (e.g., pseudoephedrine or phenylephrine) and drying agents (e.g., the antisecretory drug methscopolamine). Dextromethorphan is antitussive agent used in many over-the-counter cold medications. It has an opioid-like structure but, being a D-isomer, it does not possess the analgesic or addictiveness of opioids. It acts centrally to relieve cough, similar to opioids. It is active against dry cough and does not exhibit significant expectorant properties for productive cough. Methscopolamine nitrate is an anticholinergic and is a derivative of scopolamine. It possess the peripheral actions of the belladonna alkaloids, but does not exhibit the central actions because of its inability to cross the blood-brain barrier. In cough-cold preparations, it is most often used for its anti-secretory effect on the upper respiratory system. However, methscopolamine has a relatively short plasma half-lie and needs an extended release formulation to provide symptomatic relief over extended periods of time. Chlorpheniramine, often administered as the maleate salt, is a short-acting antihistamine (Hl) compound that also has a short plasma half-life. Chlorpheniramine also requires an extended release formulation to provide extended symptomatic relief in cough-cold formulations. Phenylephrine is an alpha agonist that acts as a decongestant in cough-cold formulations. Phenylephrine also requires a longer acting formulation. Phenylephrine hydrochloride, also known as (S)-3-hydroxy-[alpha][(methylamino) methylbenzene methanol hydrochloride, is an orally effective nasal decongestant. Phenylephrine is a synthetic, optically active sympathomimetic amine that has one hydroxyl group on the benzene ring. The hydroxyl group is meta to the aliphatic side chain. The meta position affords optimal activity. Phenylephrine (neo-synephrine) replaced an older preparation, synephrine, in which the hydroxyl was in the para position. Phenylephrine hydrochloride is available in the form of the levorotatory isomer, which is a white, odorless, non-hygroscopic, crystalline compound possessing a bitter taste. Phenylephrine hydrochloride has a melting point of 140-145° C. and is freely soluble in water and alcohol. Decongestant compounds in the form of their free bases as well as their pharmaceutically acceptable salts, e.g., hydrochloride, citrate, maleate, tannate, etc., are used. Expectorants, such as guaifenesin, change a dry, unproductive cough to one that is more productive and less frequent. Guaifenesin, known chemically as 3-(2-methoxyphenoxy)-1,2-propanediol, is a crystalline powder, which is soluble in water and alcohol. It is indicated in the USP Drug information as an expectorant for the symptomatic relief of cough due to colds and minor upper respiratory infections.

Phenylephrine may be present in amounts of between about 15 and about 60 milligrams per capsule, preferably from about 5 milligrams to about 30 milligrams per capsule, with half or less of that amount used in a pediatric form of the formulation. Delsym DM® (Novartis) is an example of a sustained-release dextromethorphan composition. The sustained-release characteristics of the composition are achieved by the use of small particles of an ion-exchange resin bound to the dextromethorphan that delay release of the drug in the gastrointestinal tract. However, the bioavailability of dextromethorphan is relatively low due to the strong bond between residual amounts of dextromethorphan and the ion exchange resin. The total percentage of dextromethorphan released from dextromethorphan-ion exchange resin complexes is incomplete, and under certain in vitro conditions it has been observed that even after a twelve hour period of time, about 20% of dextromethorphan remains bound to the ion exchange resin.

Examples of the preparation of sustained release formulations of individual cough-cold medicines include U.S. Pat. No. 6,416,786 to Mulaye (sustained release formulation of guaifenesin); U.S. Patent Application Publication No. 20050152967 by Tengler et al. (mixed release formulations of an expectorant for immediate release and a decongestant for extended release); U.S. Pat. No. 6,955,821 to Davis et al. (sustained release formulation of guaifenesin and at least one other drug, wherein guaifenesin is formulated for both immediate and extended release); and U.S. Pat. No. 6,372,252 Blume et al. (sustained release guaifenesin formulations where guaifenesin is formulated for both immediate and extended release).

While some mixed release compositions have been described, as discussed above, there is still a need in the art to create controlled release pharmaceutical formulations with better sequential release characteristics that orchestrate the symphony of symptomatic release agents to better fit the needs of patients suffering from a cold, or those experiencing cold like symptoms in terms of decongestant, expectorant and drowsiness characteristics.

Therefore it is an object of the present invention to provide a formulation with a tighter control of different drug release than what is achieved with an immediate release-sustained release formulation.

It is a further object of the invention to provide a formulation including an expectorant for immediate and extended release, and a cough suppressant for delayed and extended release, wherein the release profiles of the ingredients are orchestrated to better co-ordinate the production and clearance of mucus and cough suppression.

### SUMMARY OF THE INVENTION

A mixed-release tablet or capsule formulation including vehicles for the delivery of a plurality of drugs in various combinations of immediate release, extended release, and/or delayed release modes over a predetermined time period have been developed which provide for controlled release not only of the drugs, but controlled release that is designed to create more effective coordination between the drugs being delivered. The drugs can be any medically and/or physiologically appropriate combination of active agents and active ingredients, preferably decongestants, antihistamines, expectorants, antitussives, cough suppressants, and drying agents. Representative decongestants include phenylephrine (bitartrate, tannate, HBr, or HCl salts), phenylpropanolamine (HCl), pseudoephedrine (HCl), and combinations thereof. Representative antihistamines include chlorpheniramine (maleate), brompheniramine (maleate), dexchloropheniramine (maleate), dexbromopheniramine (maleate), triprolidine (HCl), diphenhydramine (HCl), doxylamine (succinate), tripelennamine (HCl), cyproheptatine (HCl), bromodiphenydramine (HCl), phenindamine (tartarate), pyrilamine (maleate, tamate), azatadine (maleate), and combinations thereof. Representative expectorants include guaifenesin, terpin hydrate, (glyceryl guaiacolate), potassium (iodide, citrate), potassium guaiacolsulfonate, and combinations thereof. Representative antitussive agents include caramiphen (edisylate), dextromethorphan (HBr), codeine (phosphate, sulfate), hydrocodone (bitartrate), and combinations thereof.

Appropriate vehicles for the delivery of the drugs may be liquid, gel, semisolid, paste, or solid. More specifically, these vehicles may be microparticles, for example, microspheres or other micronized forms, granules, beads, or resins. In various embodiments, the tablet or capsule pharmaceutical formulation includes one or more of the following granules: immediate-release granules, delayed release granules, extended release granules; and optionally free drug or a plurality of different drugs, wherein each formulation of granules include individual beads containing soluble and/or insoluble polymers and an active drug or a plurality of drugs. In another embodiment, the formulations include a decongestant for immediate and extended release, an antihistamine for extended release, and a drying agent for delayed and extended release, wherein the release profiles of the ingredients are controlled to orchestrate the effectiveness of their pharmacological action. For example, the formulation may comprise an expectorant for immediate and extended release, wherein over 90% of the drug is released substantially linearly over a 12 hour period of time, and a cough suppressant for delayed and extended release, wherein over 90% of the drug is released between 4 to 12 hours, wherein the release profiles of the ingredients are orchestrated to co-ordinate the production and clearance of mucus and cough suppression. In one embodiment, the pharmaceutical composition includes an extended release expectorant active drug packaged for extended release of over 90% of the expectorant active drug released substantially linearly over up to at least a 12 hour period of time; and an antitussive active drug packaged for immediate and sustained release, such that up to 50% of the antitussive active drug is released in immediate fashion over the first 1 to 4 hours, followed by sustained release of the antitussive drug, such that up to 100% of the antitussive active drug is released between 4 to 12 hours. In another embodiment, the pharmaceutical formulations include an expectorant for immediate and extended release, and a cough suppressant for delayed and extended release, wherein the release profiles of the ingredients are orchestrated to better co-ordinate the production and clearance of mucus and cough suppression.

The pharmaceutical formulation may include a combination of the anti-tussive, anti-histamine and drying agent; a combination of the decongestant and drying agent; a combination of the decongestant and cough suppressant; a combination of the expectorant and cough suppressant; or a combination of the expectorant and anti-tussive in a capsule, tablet, oral rapid dissolving tablet, softget, gelcap, film, gum, pastille, lozenge, disk, or wafer form. The antitussives, antihistamines, decongestants, expectorants, drying agents or combinations thereof may be formulated as immediate-release granules, delayed release granules, extended release granules; or optionally as a free drug depending on the desired pharmacological effect. The formulations may include effective amounts of each of dextromethorphan (HBr), chlorpheniramine (maleate), methscopolamine (nitrate), pseudoephedrine (HCl), phenylephrine (HCl), guaifenesin, and hydrocodone (bitartrate). The anti-tussive active drug can be dextromethorphan in a form such as a sustained release granule, a sustained release matrix, or combinations thereof; and the anti-histamine active drug can be chlorpheniramine in a form such as a sustained release bead, a sustained release matrix, or combinations thereof. The drying agent active drug can be methscopolamine in the form of a delayed release granule. The decongestant can be pseudoephedrine in the form of a mixture of an immediate release bead, an immediate release matrix, and a sustained release bead, a sustained release matrix, or combinations thereof.

In one embodiment, the tablet or capsule pharmaceutical formulation can include:
(a) an anti-tussive active drug within a granule matrix packaged for extended release of over 90% of the active drug released substantially linearly over up to at least a 12 hour period of time;
(b) an anti-histamine active drug packaged for extended release of over 90% of the active drug released substantially linearly over up to a least a 12 hour period of time; and
(c) a drying agent active drug packaged for delayed and extended release of over 90% of the active drug released substantially linearly over a period of 8 hours following an initial delay of release of from about 2 to about 4 hours.

In another embodiments, the pharmaceutical composition can include:
(a) an immediate release decongestant active drug packaged immediate release of over 60-80% of the active drug within from about 90 minutes to about 120 minutes following dosing;
(b) an extended release decongestant active drug packaged for extended release of over 90% of the active drug released substantially linearly over up to a 12 hour period of time; and
(c) a drying agent active drug packaged for delayed and extended release of over 90% of the active drug released substantially linearly over up to at least an 8 hour period following an initial delay of release of from about 2 to about 4 hours.

In another embodiment, the pharmaceutical composition can include:
(a) an immediate release expectorant active drug packaged for immediate release of over 60-80% of the active drug within from about 90 minutes to about 120 minutes following dosing;
(b) an extended release expectorant active drug packaged for extended release of over 90% of the active drug released substantially linearly over up to at least a 12 hour period of time;
(c) optionally, a drying agent active drug package for delayed and extended release of over 90% of the active drug released substantially linearly over up to at least an 8 hour period following an initial delay of release of from about 2 to about 4 hours; and
(d) optionally, a cough suppressant active drug packaged for delayed and extended release of over 90% of the active drug released substantially linearly over up to at least an 8 hour period following an initial delay of release of from about 2 to about 4 hours.

In another embodiment, pharmaceutical composition can include:
(a) an immediate release expectorant active drug packaged for immediate release of over 60-80% of the active drug within from about 90 minutes to about 120 minutes following dosing;
(b) an extended release expectorant active drug packaged for extended release of over 90% of the active drug released substantially linearly over up to at least a 12 hour period of time;
(c) optionally, a drying agent active drug packaged for delayed and extended release of over 90% of the active drug released substantially linearly over up to at least an 8 hour period following an initial delay of release of from about 2 to about 4 hours; and
(d) optionally, an antitussive active drug packaged for delayed and extended release of over 90% of the active drug released substantially linearly over up to at least an 8 hour period following an initial delay of release of from about 2 to about 4 hours.

In variations of these embodiments, the immediate release forms of the drugs are optional.

In a specific embodiment, the pharmaceutical composition can include:
(a) an extended release form of guaifenesin packaged for extended release of over 90% of the active drug released substantially linearly over up to at least a 12 hour period of time;
(c) hydrocodone formulated for delayed and extended release of over 90% of the active drug released substantially linearly over up to at least an 8 hour period following an initial delay of release of from about 2 to about 4 hours.

A method for providing a sequential-release formulation has also been developed, wherein different types of granules are loaded into a capsule, wherein each type of granule includes individually an extended release anti-tussive, an extended release antihistamine and a drying agent in a delayed release form, and wherein the capsule further includes one or more excipients. Alternatively, a sequential-release tablet formulation is made by compressing a mixture of excipients and a mixture of granules into a tablet, wherein each type of granule includes individually an extended release anti-tussive, an extended release anti-histamine and a drying agent in a delayed release form. The mixture of excipients may further include free active drug. The anti-tussive agent can be dextromethorphan, the anti-histamine can be chlorpheniramine, and the drying agent can be methscopolamine.

Another method for making a sequential-release formulation includes loading a mixture of different types of granules and an excipient formulation into a capsule, wherein the mixture of granules includes an active drug within a polymer matrix and wherein the active drug mixture includes an immediate release decongestant, an extended release decongestant, and a drying agent in a delayed and extended release form. The anti-tussive agent can be dextromethorphan, the anti-histamine can be chlorpheniramine, and the drying agent can be methscopolamine.

Still another method for making a sequential-release formulation includes loading a mixture of different types of granules into a capsule, wherein each type of granule includes individually an extended release expectorant, and a cough suppressant in a delayed and extended release form, and wherein the capsule includes one or more excipients.

In one embodiment, the expectorant can be guaifenesin and the cough suppressant can be hydrocodone. In one embodiment of a formulation of hydrocodone and guaifenesin, up to 20% of the hydrocodone can be released in a sustained fashion over the first 1 to 4 hours, followed by the sustained release of up to 100% of the remaining amount of the hydrocodone between 4 to 12 hours.

In another formulation of hydrocodone and guaifenesin, the release of hydrocodone is achieved in two sequential immediate release pulses, where between 0 to 5 mg of hydrocodone is released between 1 to 4 hours, and 5 to 10 mg of the hydrocodone is released after 4 to 6 hours. In yet another formulation of hydrocodone and guaifenesin, 20-35% of guaifenesin is released between 0 to 2 hours, 35-65% is released between 2 to 4 hours, and 65-90% is released between 4 to 8 hours. In another formulation of hydrocodone and guaifenesin, up to 50% of the hydrocodone (0-5 mg) is released between 1 to 4 hours, and 5 to 10 mg of the hydrocodone is released in a sustained fashion between 4 to 12 hours. In this specific formulation guaifenesin is released in a sustained fashion between 1-12 hours.

### DETAILED DESCRIPTION OF THE INVENTION

Definitions

As used herein, “solid pharmaceutical compositions” include, but are not limited to, oral dosage forms such as tablets, capsules, caplets, gelcaps, wafers, films, gums, pastilles, lozenges, matrices, disks. Such compositions are predominantly solid such that appropriate dosages are achieved by the subject without the need for a separate measuring device, such as a spoon, measuring cup or dropper. Such compositions can include liquid constituents or compositions, including gels and slurries.

As used herein “delayed release” means that about 90% or more of the active therapeutic agent administered and contained within the formulation, for example within granules, is delayed for about 1 to 4 hours, more precisely, 2 to 4 hours, after administration, from being released from the granules and available to systemic exposure.

As used herein “sustained release” or “extended release” means that about 90% or more of the active therapeutic agent administered and contained within the formulation, for example, within granules, is released from the granules and available to systemic exposure over there period of time where release is sustained and released over a substantially linear rate.

As used herein “immediate release” means that at about 90% or more of the active therapeutic agent administered and contained within the formulation, for example, within granules, is release within a period of 1 to 3 hours after administration and available to systemic exposure. Immediate release may also be defined functionally as the release of over about 80 to 90 percent 15 (%) of the active ingredient within about 60, 90, 100 or 120 minutes or less.

As used herein, a “pulse” relates to a release of the active therapeutic agent at a contemplated point in time. For example, a pulse can be delayed, but once the point in time is reached following administration, the active therapeutic agent can be released immediately. Pulses can be sequential. For example, a first immediate release pulse can occur at time point X, which is followed later by a second immediate release pulse at time point Y. Although commencement of the second immediate release pulse is delayed in comparison to the commencement and possibly the completion of first time pulse, the release of the second will be immediate following the delay.

The term “about” in the context of numerical values and ranges refers to values or ranges that approximate or are close to the recited values or ranges such that the method or composition can perform as intended, as is apparent from the teachings contained herein. Thus, this term encompasses values beyond those simply resulting from systematic error. Typically, the term “about” means plus or minus ten percent.

### I. Formulation

The formulations are based on the recognition that patients and physicians are looking to simplify the number of doses that a patient takes and to improve the timing of onset of drug action consistent with improving patient compliance and outcomes. The underlying rational is the use of combinations of drugs in a single therapeutic preparation, where each active agent or drug, or groups of drugs is formulated in a specific release profile so as to provide in combination with the other actives agents to provide a sequence of complementary therapeutic actions. The therapeutic utility of the formulation is a result of orchestrating the overall utility of the therapeutic preparation over time. For example, a therapeutic formulation that is formulated to be taken in the early evening provides an immediate dose of guaifenesin that can assist in breaking up mucus and initiating a productive cough. As the guaifenesin action decreases, the drying and decongestant activities of a selectively delayed and controlled release methscopolamine and phenylephrine provides longer term drying and decongestant activity and which is less likely to interfere with the user's sleep.

Methscopolamine and phenylephrine can delayed for about 2-4 hours after administration and only released so as to provide a sequence of mucolytic and then decongestant and drying effects better fitted with the patient's needs. Patients who have experienced an exposure to an allergen benefit from a rapid release of the antihistamine chlorpheniramine but, one removed from the presence the allergen, better benefit from the sustained release of a decongestant combined with the longer term drying effects of methscopolamine, the gradual controlled release of which is delayed 2-4 hours after dosing. By this time, the immediately released chlorpheniramine effects are declining and are supplemented by a sustained drying effect provided by methscopolamine and a decongestant effect provided by phenylephrine. This example also illustrates ways in which the combination of delayed, controlled and immediate release actives can be blended to avoid side effects found in some combinations of less skillfully regulated blends of actives. Chlorpheniramine and methscopolamine are highly effective in their own regard. For example, while both chlorpheniramine and methscopolamine are highly effective, both have anticholinergic activity which, when combined as in a simple controlled release preparation containing both actives, causes excessive and uncomfortable drying in some patients. However, by combining a release profile for chlorpheniramine that sequentially provides relief from the different actives, a therapeutic is created that is more effective in addressing both the short term and long term therapeutic needs of patients while avoiding unpleasant side effects resulting from less precise delivery profiles.

A. Pharmaceutically Active Compounds

The drugs may be any medically and/or physiologically appropriate combination of drugs and active ingredients described in United States Pharmacopoeia (“USP”), the Physician's Desk Reference (“PDRW”), and the Food and Drug Administration (“FDA”) publication, “Approved Drug Products with Therapeutic Equivalence Evaluations,” also known as the “Orange Book.” The drugs can be any medically and/or physiologically appropriate combination of drugs and active ingredients, preferably decongestant drugs, antihistamines, expectorants, antitussives, cough suppressants, and drying agents. Representative decongestant active drugs include phenylephrine (bitartrate, tannate, HBr, HCl), phenylpropanolamine (HCl), pseudoephedrine (HCl), and combinations thereof. Representative antihistamine active drug include chlorpheniramine (maleate), brompheniramine (maleate), dexchloropheniramine (maleate), dexbromopheniramine (maleate), triprolidine (HCl), diphenhydramine (HCl), doxylamine (succinate) tripelennamine (HCl), cyproheptatine (HCl), bromodiphenhydramine (HCl), phenindamine (tartarate), pyrilamine (maleate, tamate), azatadine (maleate), and combinations thereof. Representative expectorant active drug include guaifenesin, terpin hydrate, (glyceryl guaiacolate), potassium (iodide, citrate), potassium guaiacolsulfonate, and combinations thereof. Representative antitussive active drug include caramiphen (edisylate), dextromethorphan (HBr), codeine (phosphate, sulfate), hydrocodone (bitartrate), and combinations thereof. Representative cough suppressants include benzonatate, bromodiphenhydramine, brompheniramine, buclizine, carbinoxamine, chlorpheniramine, clemastine, codeine, cyclizine, dexchloropheniramine, dextromethorphan, dihydrocodeine, dimenhydrinate, diphenhydramine, doxylamine, homatropine, hydrocodone, meclizine, pheniramine, phenyltoloxamine, pyrilamine, trimethobenzamide, triprolidine. Representative drying agents include methscopolamine, scopolamine, meclizine, trimethobenzamide, dimenhydrinate, cyclizine, pheniramine, buclizine, chlorpheniramine, diphenhydramine, homatropine, carbinoxamine, clemastine, brompheniramine, and doxylamine.

Effective Dosages are known.

B. Excipients

Appropriate vehicles for the delivery of the drugs may be liquid, gel, semisolid, paste, or solid. More specifically, these vehicles may be microparticles, for example, microspheres or other micronized forms, granules, beads, or resins. In various embodiments, the tablet or capsule pharmaceutical formulation includes one or more of the following granules: immediate-release granules, delayed release granules, extended release granules; and optionally free drug or a plurality of different drugs, wherein each formulation of granules includes individual beads containing soluble and/or insoluble polymers and an active drug or a plurality of drugs. In another embodiment, the formulations include a decongestant for immediate and extended release, an anti-histamine for extended release, and a drying agent for delayed and extended release, wherein the release profiles of the ingredients are controlled to orchestrate the effectiveness of their pharmacological action. In another embodiment, the inventive pharmaceutical formulations include an expectorant for immediate and extended release, and a cough suppressant for delayed and extended release, wherein the release profiles of the ingredients are orchestrated to better coordinate the production and clearance of mucus and cough suppression.

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes, but is not limited to, diluents, binders, lubricants, desintegrators, fillers, and coating compositions.

“Carrier” also includes all components of the coating composition which may include plasticizers, pigments, colorants, stabilizing agents, and glidants. The delayed release dosage formulation may be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6.sup.th Edition, Ansel et al., (Media, Pa.,: Williams and Wilkins, 1995) which provides information on carriers, material, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit®. (Roth Pharma, Westerstadt, Germany), Zein, shellac, and polysaccharides. Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

Diluents, also termed “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethyl cellulose, hydroxypropylcellulose, ethylcelluose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked polyvinylpyrrolidone (Polyplasdone® XL from GAF Chemical Corp).

Stabilizers are used to inhibit or retard drug decomposition reactions which include, by way of example, oxidative reactions.

Surfactants may bee anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

### Extended Release Dosage Forms

The extended release formulations are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene, Hydrophilic polymers include, but are not limited to, methylcellulose, hydroxypropylcellulose, hydroxypropylmethylcellulose, sodium carboxymethylcellulose, and Carbopol® 934, polyethylene oxides. Fatty compounds include, but are not limited to, various waxes such as a carnauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic slats such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

### Delayed Release Dosage Forms

Delayed release formulations are created by coating a solid dosage form with a film of polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art of evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizer include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles i the dispersion. Typical stabilizing agents are nonionic emulsifiers. such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be use. Small quantities of an anti-foaming agent, such as a silicone (e.g., simethicone), may also be added to the coating composition.

As will be appreciated by those skilled in the art and as described in the pertinent texts and literature, a number of methods are available for preparing drug-containing tablets, beads, granules or particles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with a suitable complexing agent.

The delayed release dosage units may be coated with the delayed release polymer coating using conventional techniques, e.g., using a conventional coating pan, an airless spray technique, fluidized bed coating equipment (with or without a Wurster insert), or the like. For detailed information concerning materials, equipment and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6.sup.th Ed. (Media, Pa.,: Williams & Wilkins, 1995).

### Sustained Release Formulations

Sustained release matrices are prepared from mixtures of soluble polymers such as ethylcellulose, cellulose acetate, vinyl acetatelvinyl chloride copolymer, acrylate-methacrylate copolymers, polyethylene oxide, hydroxypropyl methylcellulose, carageenan, alginic acid and salts thereof, hydroxyethyl cellulose, hydroxypropyl cellulose, karaya gum, acacia gum, tragacanth gum, locust bean gum, guar gum, sodium carboxymethyl cellulose, methyl cellulose, beeswax, carnauba wax, cetyl alcohol, hydrogenated vegetable oils, stearyl alcohol; and combinations thereof. Carbohydrate particles (non-pareils) can be used as inert cores or carriers in capsule and tablet formulations and are coated by active ingredients. These particles are usually spherical and contain sucrose (62.5%-91.5%) and starch.

Ion exchange resins suitable for use in granule components are water-insoluble and consist of a pharmaceutically inert organic or inorganic matrix containing covalently bound functional groups, wherein the functional groups are ionic or capable of being ionized under the appropriate conditions of pH. The organic matrix may be synthetic (e.g., polymers or copolymers of acrylic acid, methacrylic acid, sulfonated styrene, sulfonated divinylbenzene), or partially synthetic (e.g., modified cellulose and dextrans). The inorganic matirx can also be, for example, silica gel modified by the addition of ionic groups. The covalently bound ionic groups may be strongly acidic (e.g., sulfonic acid), weakly acidic (e.g., carboxylic acid), strongly basic (e.g., quaternary ammonium), weakly basic (e.g., primary mine), or a combination of acidic and basic groups. In general, those types of ion exchangers suitable for use in ion exchange chromatography and for such applications as deionization of water are suitable for use in the granule component. Such ion exchangers are described by H. F. Walton in “Principles of Ion Exchange” (pp. 312-343), incorporated by reference herein. Useful ion exchange resins generally have exchange capacities below about 6 milliequivalents per gram (meq/g) and preferably below about 5.5 meq/g. The granule ion-exchange resin is cross-lined with a crosslinking agent selected from difunctional compounds capable of cross-linking polystyrenes. The cross-linking agent can be a divinyl or polyvinyl compound. The cross-linking agent can be divinylbenzene. The resin is cross-linked to an extent of about 3 to about 20%, preferably about 4 to about 1696, more preferably about 6 to about 1o%, and most preferably about 8% by weight based on the total resin. Representative granule resins include Amberlite® IRP-69 (Rohm and Haas) and Dow XYS®-40010.00 (Dow Chemical Company). Both are sulfonated. polymers composed of polystyrene cross-linked with 8% of divinylbenzene, with an ion exchange capacity of about 4.5 to 5.5 meq./g of dry resin (Ht-form). Their essential difference is in physical form. Amberlite® 1W-69 consists of irregularly-shaped particles with a size range of 47 to 149 um, produced by 15 milling the parent, large-sized spheres of Amberlite® IRP-120. The Dow XYS8-40010.00 produce consists of spherical particles with a size range of 45 to 150 um. Another useful exchange resin, Dow XYS®-40013.00, is a polymer composed of polystyrene cross-linked with 8% of divinylbenzene and functionalized with a quaternary ammonium group; its exchange capacity is normally within the range of approximately 3 to 4 meq/g of dry resin.

U.S. Pat. Nos. 2,990,332 to Keating and 4,221,778 to Raghunathan, describe adsorption techniques of active drug agents onto ion exchange resin particles to form the drug-resin complex. Briefly, the drug is mixed with an aqueous suspension of the resin, and the complex is then washed and dried. Adsorption of drug onto the resin may be detected by measuring a change in the pH of the reaction medium, or by measuring a change in concentration of sodium or drug. Binding of drug to resin can be accomplished according to four general reactions. In the case of a basic drug, these are: (a) resin (Na-form) plus drug (salt form); (b) resin ma-form) plus drug (as free base); (c) resin (H-form) plus drug (salt form); and (d) resin (H-form) plus drug (as free base). All of these reactions except (d) have cationic by-products, by completing with the cationic drug for binding sites on the resin, reduce the amount of drug bound at equilibrium. For basic drugs, stoichiometric binding of drug to resin is accomplished only through reaction (d). Four analogous binding reactions can be carried out for binding an acidic drug to an anion exchange resin. These are: (a) resin (el-form) plus drug (salt form); (b) resin (Cl-form) plus drug (as free acid; (c) resin (OH-form) plus drug (salt form); and (d) resin (OH-form) plus drug (as free acid). For acidic drugs, stoichiometric binding of drug to resin is accomplished only through reaction (d). The binding may be performed, for example, as a batch or column process, as is known in the art. The drug-resin complex formed is collected and washed with ethanol and/or water to insure removal of any unbound drug. The complexes are usually air-dried in trays at room or elevated temperature. The drug-resin complex has a ratio of active drug(s) to resin of about 1:3 to about 3:1, preferably about 1:2 to about 2:1, most preferably about 1:1 (by weight).

C. Formulations

The devices with different drug release mechanisms described above can be combined in a final dosage form comprising single or multiple units. Examples of multiple units include multilayer tablets, capsules containing tablets, beads, granules, etc. An immediate release portion can be added to the extended release system by means of either applying an immediate release layer up top of the extended release core using coating or compression process or in a multiple unit system such as a capsule containing extended and immediate release beads.

In one embodiment, a tablet or capsule pharmaceutical formulation includes an anti-tussive agent in an immediate release form, and anti-histamine in an extended release form and a drying agent in a delayed and extended release form.

In another embodiment, the composition includes a decongestant packed for immediate release; a decongestant packed for extended release, and a drying agent packed for delayed and sustained release, wherein the decongestant provides immediate decongestant relief followed by long-acting decongestant activity, wherein the release of the drying agent is delayed to allow initial decongestion followed by long-acting drying agent activity.

In another embodiment, a time-release dextromethorphan and a timed release chlorpheniramine are formulated to provide maximum effective release over up to at least 12 hours or more using a combination of polymers in the bead matrix or coat. The drying agent methscopolamine is formulated to provide a 2 to 4 hour delay of release followed by maximum effective release between 4 to 8 hours using a combination of polymers in the bead matrix and coating materials such as pH sensitive polymers or shellac.

### Methods of Manufacture

A preferred method of preparing an extended release tablet is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

Active pharmaceutical ingredients (API's) can be processed by agglomeration, air suspension drying, balling, coacervation, coating, comminution, compression, cryoplelletization, encapsulation, extrusion, wet granulation, dry granulation, homogenization, inclusion complexation, lyophilization, melting, microencapsulation, mixing, molding, pan coating, solvent dehydration, sonication, spheronization, spray drying or other processes known in the art to form granules.

In a wet granulation method, the at least one pharmaceutically active agent and other ingredients are granulated with a granulating fluid (e.g., isopropyl alcohol, ethyl alcohol, and water) in a planetary mixer, high shear mixer, or fluidized bed granulator. Binding agents may be contained in the granulating fluid, or may be in the dry mix. The wet granules are dried in an oven or fluidized bed dryer, and then sieved through a suitable screen to obtain free flowing granules. The resulting granules are blended with a suitable lubricant and glidant, and the lubricated granules are compressed into tablets on a rotary press using appropriate tooling. If desired, a coating can be applied onto a rotary press using appropriate tooling. If desired, a coating can be applied onto the compressed tablets.

Typical excipients used in these methods include bulking agents, disintegrating agents, anti-adherents and glidants, lubricants, and binding agents. Bulking agents include, but are not limited to, microcrystalline celluose (e.g., Avicel®Q, FMC Corp.; Emcocel®Q, Mendell Inc.), mannitol, xylitol, dicalcium phosphate (e.g., Emcompress®, JRS Pharma) calcium sulfate (e.g., Compactrol®, JRS Pharma) starches, lactose, sucrose (Dipac, Amstar, and Nutab, Ingredient Technology), dextrose, sorbitol, cellulose powder (Elcema, Degussa, and Solka Floc, JRS Pharma). The bulking agent may be present in the pharmaceutical composition in an amount of from about 5 wt. % to about 90 wt. % preferably from about 10 wt. % to about 50 wt. %. Disintegrating agents may be included in the pharmaceutical formulation and include, for example, microcrystalline cellulose, starches, crospovidone (e.g., Polyplasdone XL®, International Specialty Products.), sodium starch glycolate (Explotab®, JRS Pharma), and crosscarmellose sodium (e.g., Ac-Di-Sol®, FMC Corp.). The disintegrating agent may be present in the composition in an amount of from about 0.5 wt. % to about 30 wt. %, preferably from about 1 wt. % to about 20 wt. %. Antiadherants and glidants may be used in the pharmaceutical composition include, for example, talc, corn starch, silicon dioxide, sodium lauryl sulfate, and metallic stearates. The antiadherent or glidant may be present in the pharmaceutical formulation in an amount of from about 0.2 wt. % to about 15 wt. %, preferably from about 0.5 wt. % to about 5 wt. %.

Examples of lubricants used in the excipient mixture include magnesium stearate, calcium stearate, sodium stearate, stearic acid, sodium stearyl fumarate, hydrogenated cotton seed oil (sterotex), talc, and waxes, including but not limited to, beeswax, carnauba wax, cetyl alcohol, glyceryl stearate, glyceryl palmitate, glyceryl behenate, hydrogenated vegetable oils, and stearyl alcohol. The lubricant may be present in an amount of from about 0.2 wt. % to about 20 wt. %, preferably from about 0.5 wt. % to about 5 wt. %. Binding agents include, for example, polyvinyl pyrrolidone, starch, methylcellulose, hydroxypropyl methylcellulose, carboxymethyl cellulose, sucrose solution, dextrose solution, acacia, tragacanth and locust bean gum. The binding agent may be present in the composition in an amount of from about 0.2 wt. % to about 10 wt. %, preferably from about 0.5 wt. % to about 5 wt. %. Granules (coated or uncoated) containing active agents are combined with excipients and compressed into a table formulation.

Extended release and delayed release formulations may be prepared and delivered so that release is accomplished in some generally predictable location in the lower intestinal tract more distal than would have accomplished if there had been no delayed release alterations. A method for delay of release is a polymeric coating on each granule intended for extended or delayed release in a more distal portion of the gastrointestinal tract. Any coatings should be applied in sufficient thickness such that the entire coating does not dissolve in the gastrointestinal fluids at pH below about 5, but does dissolve at pH 5 and above. It is expected that any anionic polymer exhibiting a pH-dependent solubility profile can be used as an enteric coating in the practice of the present invention to achieve delivery to the lower gastrointestinal tract. Polymers and compatible mixtures thereof may be used to provide the coating for the delayed or the extended release of active ingredients. By way of an example, the sustained release of dextromethorphan, chlorpheniramine, and methscopolamine, over up to at least a 12 hour or more period, was achieved through formulation in a matrix of hydroxyproplymethylcellulose (Methocel® K100M Premium USP Powder), dicalcium phosphate dihydrate, crosscannellose sodium, silicon dioxide, microcrystalline cellulose pH 102 and magnesium stearate. Sustained release granules of methscopolamine, prepared from the same excipients can be coated with pH sensitive polymers that degrade in the lower Gl tract, (e.g., Eudragit® L. Eudragit® S, Aquot AS-HF, and ethylcellulose) so that release of active is delayed for 2 to 4 hours until the granule reached the distal ileum colon. The length of the day is controlled by the thickness of coating (amount). Granules can also be coated with shellac. The resulting mixture of granules and excipients can be either compressed into a tablet or filled into a capsule.

In one embodiment, the compositions include drug-resin complexes having two or three active ingredients. In another embodiment, the invention also provides pharmaceutical compositions including the drug-resins in combination with suitable pharmaceutically acceptable excipients, and optionally at least one of an antihistamine, sympathomimetic drug (nasal decongestant, bronchodilator), analgesic, antiinflammatory, cough suppressant and/or expectorant. The mean particle size of the granule ion-exchange resins is within the range of about 100 to about 300 pm.

In another embodiment pharmaceutical combinations include extended release granules of anti-histamines, decongestants, are mixed with delayed and sustained release formulations of drying agents and compressed into rapid dissolving oral tablets. The tablet can further include a binder to permit particles of the porous, plastic substance and the water penetration enhancer to adhere to each other sufficiently to prevent particle segregation and to increase granule adherence at the low pressure used to form the tablet. The binder can include from about 1% to about 90% of the tablet by weight. For example, the binder can be fructose or mannose. The tablet can further include at least one additional ingredient selected from the group consisting of a surfactant, superdisintegrant, superporous hydrogel particle, effervescent agent, lubricant, flavoring agent, a coloring agent and combinations thereof. Some formulations use effervescent couples as their disintegrant, while others use a combination of disintegrants. U.S. Pat. No. 6,596,311 describes different types of non-effervescent disintegrants.

The formulation can include three active drugs, each within its own granule composition or within the tablet or capsule matrix, wherein the active drugs are an anti-tussive, an antihistamine, and a drying agent. For example, from about 5% to about 20% of the total active anti-tussive is contained within the polymeric material and from about 80% to about 95% is packaged within the extended release granules polymeric material. For example, from about 5% to about 20% of the drying agent is release over the first four hours after dosing and from about 80% to about 95 percent is released in the period from 4 hours to 24 hours after dosing. For example, from about 5% to about 20% of the total active antihistamine is contained within the rapid dissolve polymeric material and from about 80% to about 95% is packaged within the extended release granules polymeric material. The active anti-tussive can be dextromethorphan. The active antihistamine can be chlorpheniramine. The drying agent can be methscopolamine. The decongestant can be phenylephrine or pseudoephedrine.

### EXAMPLE 1

### Sustained Release Table of Dextromethorphan.

This example provides a sustained release tablet of dextromethorphan, chlorpheniramine and methscopolamine. The tablet was prepared by direct compression of the ingredients listed in Table 1:

TABLE 1
TABLET FORMULATION

| Component | Weight Tablet (mg) |
|---|---|
| Dextromethorphan hydrobromide | 30.60 |
| Chlorpheniramine maleate | 8.16 |
| Methscopolamine nitrate | 2.55 |
| Dicalcium phosphate dehydrate | 0.50 |
| Croscarmellose sodium (powder) | 22.50 |
| Methocel ® (K100M Premium USP powder) | 37.50 |
| Silicon Dioxide | 5.00 |
| Microcrystalline cellulose PHI02 | 380.69 |
| Magnesium stearate | 12.50 |

This formulation achieved a sustained release of all actives approximately linearly over a 12 hour period. In formulation that require a delayed release of methscopolamine, sustained release granules of methscopolamine, prepared using excipients shown in Table 1, were coated (10-65% by weight), with the water insoluble and pH sensitive polymers, Eudragit® RS or Eudragit® RL, or combination thereof, using a Wurster Fluidiized Bed process. Coated granules of methscopolamine were blended with the other actives and either directly compressed into tablets or filled into capsules.

### EXAMPLE 2

### Sustained Release Beads of Guaifenesin

Sustained release beads of guaifenesin were prepared. Briefly, guaifenesin was first granulated with 3% 90F Kollidon® and 0.2% Cab-o-Sil, dried and the mixture passed through a 60 20 mesh screen to collect 200 μm particles. The particles were then placed in a Surster Fluidized Bed coating machine and coated with a latex dispersio of ethyl cellulose (SURELEASE®, Colorcon). Coated granules were air dried. The ingredients of the coated granules are listed in Table 2 below:

TABLE 2

| | Component % (by weight) |
|---|---|
| Guaifenesin | 58.7 |
| SURELEASE ® | 39.4 |
| 90F Kollidon ® | 1.8 |
| Cab-o-Sil | 1.0 |

Dissolution tests were then carried out on the granules of this example using a standard USP 23NF Drug Release Apparatus filled with 999 ml of the dissolution medium in the vessel and equilibrated to 37° C.±0.5° C. The paddles were set to rotate at 60 RPM. 1 G of granules was added to each vessel. The percent dissolution was determined by HPLC and the results shown in Table 3 below:

TABLE 3

| | Time (h) |
|---|---|
| 1 | 51.2 |
| 2 | 73.6 |
| 3 | 86.1 |
| 4 | 93.3 |
| 5 | 97.0 |
| 6 | 99.3 |
| 7 | 100.1 |

### Additional coating with SURELEASE® prolonged the sustained release of guaifenesin to about 12 hours.

In formulations that require a delayed release of hydrocodone, the drug plus the sustained release excipients, shown in Table 2, were granulated with 3% 90F Kollidon and 0.2% Cab-o-Sil, sized and then dried. The particles were then coated (−10-65% by weight) with water insoluble polymers Eudragit® RS or Eudragit® RL or combinations thereof, using a Wurster Fluidized Bed coating machine. These granules were then blended with guaifenesin granules and directly compressed into tablets.

### EXAMPLE 3

### Oral Rapid Dissolving Tablets

This example illustrates the making of an oral rapid dissolving tablets prepared from sustained release granules containing chlorpheniramine maleate (8 mg) and phenylephrine HCl (20 mg), and methscopolamine nitrate (2.5 mg). Chlorpheniramine maleate, 90K Kollidon® and Cab-o-sil were mixed and then coated with SURELEASE® to form the first sustained release granules. Delayed release (second) granules having methscopolamine as the active drug and containing 90F Kollidon, Cab-041 were coated with Eudragit® RS or Eudragit® RL or combinations thereof. The coated granules achieved a delayed release of methscopolamine for about 2-4 hours compared to the other actives. Mixtures of first sustained release and second delayed release granules were combined with a fast-dissolving excipient systems, (F-Melt, Fuji Chemical Industry) or Pharmaburst™ (SPI Pharmaceuticals), and then directly compressed into tablets. These tablets dissolved in the oral cavity within 30 seconds. Typically, the final rapid dissolve formulation contained fast-dissolving excipients (−40-70% by weight), sustained and delayed release granules containing actives (−30-60% by weight) and magnesium stearate (−0.4%)

### EXAMPLE 4

### Sustained Release Beads or Granules

A capsule was prepared containing sustained release beads or granules of chlorpheniramine maleate and phenylephrine HCl. Delayed release beads or granules of methscopolamine nitrate were added to the capsule formulation.

### Formulation 1:

Chlorpheniramine maleate (30 mg), phenylephrine HCl (15 mg), methscopolamine nitrate (1.25-2.5 mg), non-pareil beads (Sugar Spheres NF of 16-18 mesh cut), Shellac, Eudragit® S, Eudragit® L. Chlorpheniramine maleate and phenylephrine HCl were individually transferred to a coating pan and added to non-pareil beads using Shellac, and allowed to roll and cure for 6 hours before sustained release coating was added. The beads were coated with different amounts of Shellac (ratio of bed mix: Shellac of 1.5:1-2:1) to achieve a sustained release of active for up to 12 hours. After coating the coated beads were at 40° C.

Beads loaded with methscopolamine were coated together with a first layer of Shellac and then coated again by an outer layer(s) of a mixture of Eudragit® S and Eudragit® L. This results in a delayed release of active drugs for 2 to 4 hours followed by a sustained release.

### Formulation 2:

Chlorpheniramine maleate (30 mg), phenylephrine HCl (15 mg), methscopolamine nitrate (1.25-2.5 mg), dicalcium phosphate dehydrate, croscmnellose sodium, Methocel® K100M premium USP powder, ProsolvCD® (silicilized HPMC; JRS Pharma), magnesium stearate, Eudragit® S, Eudragit® L are provided in a formulation to first form mini-tabs (Mini Tab Press, SMI) and then load the mixture of mini-tabs into capsules. Chlorpheniramine maleate and phenylephrine HCl are blended with the other excipients and compressed into mini-tabs (2 mm in length). Methscopolamine nitrate is separately blended with excipients including, croscarmellose sodium, Methocel® K100M, magnesium stearate, and Prosolv®, and the blend is compressed into 5 mini-tabs. The mini-tabs are coated with Eudragit® S and Eudragit® L to give a delayed release. Mini-tabs (2 mm in length) are loaded into capsules. Prosolv@ is used for the compression of mini-tabs.

### Formulation 3: Chlorpheniramine Delayed and Sustained Release Tablets

Chlorpheniramine maleate (30 mg), phenylephrine HCl (15 mg), methscopolamine nitrate (1.25-2.5 mg), dicalcium phosphate dehydrate (0.5 mg), croscarmellose sodium (22.5 mg), Methocel® K100M premium USP powder (37.5 mg), silicon dioxide (5 mg), microcrystalline cellulose PH102 (380 mg), magnesium stearate (12.5 mg), Eudragit® S, Eudragit® L, are used in a formulation of tablets having both delayed release and sustained release characteristics. Chlorpheniramine maleate and phenylephrine HCl are blended with the above excipients. Methscopolamine nitrate is separately blended with the above excipients and granulated. After drying, the methscopolamine nitrate granules are coated with Eudragit® S/L using a Wurster Fluidized Bed coating machine. The resulting coated methscopolamine granules are introduced into the chlorpheniramine maleate/phenylephrine HCl blend and compressed into tablets. The resulting formulation can achieve a sustained release of chlorpheniramine maleate and phenylephrine HCl over a 12 hours period and a sustained release of methscopolamine nitrate after an initial 2-4 hours delay.

### EXAMPLE 5

### Sustained Release Formulation

This example describes a sustained release formulation of a decongestant (guaifenesin) and a cough suppressant (hydrocodone bitartrate). The formulation consists of 1000 mg of guaifenesin, and 10 mg of hydrocodone bitartrate. The formulation is manufactured as a matrix tablet made by direct compression, and contains no immediate release formulation of either guaifenesin or the hydrocodone bitartrate. The tablet is formulated so as to release guaifenesin and hydrocodone over a period of 8 to 12 hours after ingestion. The formulation includes the following standard excipients as shown in Table 4.

TABLE 4

Dicalcium phosphate dihydrate
Croscarmellose sodium
Methocel ® K100M
Silicon Dioxide
Microcrystalline cellulose PH 102 (sustained release component)
Magnesium stearate

### EXAMPLE 6

### Immediate and Sustained Release Formulation

This example describes a sustained release formulation of a decongestant (guaifenesin) and a cough suppressant (hydrocodone bitartrate) with an immediate release cough suppressant (hydrocodone bitartrate) component. The sustained release formulation core consists of 1000 mg of guaifenesin, and 5 mg of hydrocodone bitartrate. The formulation is manufactured as a matrix tablet made by direct compression, and contains no immediate release formulation of either guaifenesin or the hydrocodone bitartrate. The tablet is formulated so as to release guaifenesin and hydrocodone over a period of 8 to 12 hours after ingestion. The formulation includes the following standard excipients:
Dicalcium phosphate dihydrate
Croscarmellose sodium
Methocel® K100M
Silicon Dioxide
Microcrystalline cellulose PH 102 (sustained release component)
Magnesium stearate

The sustained release core is coated with an immediate release film containing 5 mg cough suppressant (hydrocodone bitartrate) by processes readily known to those skilled in the art.

### EXAMPLE 7

### Sustained Release Granules

This example provides a matrix tablet of sustained release granules, in the form of bead manufactured by direct compression that provides a release profile of sustained release of guaifenesin and hydrocodone over a period of 8 to 12 hours after ingestion. The formulations are designed such that 10 to 20% of the hydrocodone is released within the first 4 hours after ingestion, and 80 to 90% of the hydrocodone is released between 4 to 12 hours after ingestion. There are no separate immediate release guaifenesin components in the formulations. Variation in the properties of the guaifenesin and hydrocodone formulations are achieved by adjusting the composition and thickness of the granule coatings and the composition of the tableting excipients to vary the release profile of sustained release guaifenesin followed by a sustained release of hydrocodone after the initial delay of approximately 4 hours.

### Formulation 1:

Formulation 1 can be manufactured by first wet mixing (granulation) guaifenesin and hydroxypropyl methylcellulose, in a high-energy, high-shear mixer to produce dense guaifenesin pellets. In a similar process hydrocodone and hydroxypropyl methylcellulose is mixed to produce dense hydrocodone pellets. The resulting guaifenesin and hydrocodone pellets are sieved to the same size. The pellets are then combined and mixed with more hydroxypropyl methylcellulose and compressed into tablets. The resulting tablets are then formed by a mixture of hydroxypropyl methylcellulose-guaifenesin granulation, hydroxypropyl methylcellulose-hydrocodone granulation and additional hydroxypropyl methylcellulose.

Once the formulations are swallowed or consumed, the tablets become wet and the hydroxypropyl methylcellulose surrounding the tablets is believed to form thin gel layers. Any granular guaifenesin exposed to the exteriors of the tablets will dissolve out of the tablets, resulting in an intermediate rate of guaifenesin for absorption. As the guaifenesin leaves the outer surfaces of the tablets, gastrointestinal fluid can reach deeper into the tablets, resulting in thicker gel layers and the dissolution of the intermediate release guaifenesin granules surrounded by the gel layers. The gel layers then act as controlled release layers for dissolved guaifenesin originating in the intermediate release guaifenesin granules. Erosion of the gel layers results in further drug release. Hydrocodone is released in a similar fashion from hydrocodone granules.

Additional polymers such as hydroxypropyl methylcellulose of differing molecular weights or ethylcellulose in the hydrocodone granules are added to delay the release of hydrocodone for approximately 4 hours. An exemplary swelling agent is hydroxypropyl methylcellulose, in an amount ranging from about 5% to about 50% parts by weight per 100 parts by weight of tablet or formulation.

The formulation ensures sustained time release over a period of approximately 8 to 12 hours as demonstrated by in vitro dissolution techniques known to those skilled in the art. A binder may also be employed, preferably a material such as one or more of a group of polymers having the repeating unit of 1-ethenyl-2-pyrrolidinone. These polymers generally have molecular weights of between about 10,000 and 700,000 Daltons, and are also known as “povidone”. Amounts of the binder material will vary depending upon the nature of the binder and the amount of other ingredients of the compositions. An exemplary amount of povidone is from about 1% to about 5% by weight of povidone per 100 parts by weight of the total formulation. Processing aids such as lubricants, including stearic acid, may also be employed, as is known in the art. An exemplary amount of stearic acid is from about 0.5% to about 2.0% by weight per 100 parts by weight of table or formulation. One composition of formulation 1 is provided in Table 4:

TABLE 4

| Tablet Composition | Ingredient Weight (mg) |
|---|---|
| Guaifenesin | 1000 |
| Hydrocodone | 10 |
| Hydroxypropyl methylcellulose | 406 |
| Povidone | 35 |
| Stearic Acid | 15 |
| TOTAL | 1466 |

Granulation Process Description

The unprocessed guaifenesin and hydrocodone raw materials are dispensed and granulated in a high shear granulator. The wet granules are sieved into a fluid bed drier ad dried. When the drying process is complete, the granules are milled. Milling ensures uniform particle size distribution throughout the guaifenesin and hydrocodone granules.

Tablet Process Description

The guaifenesin and hydrocodone tablet blend is manufactured by blending the guaifenesin and hydrocodone granulations, extragranular Methocel® E10M and Hystrene® 5016 (Stearic acid). A guaifenesin and hydrocodone tablet blend is compressed to form guaifenesin and hydrocodone tablets.

Manufacturing Process

The guaifenesin and hydrocodone tablets may be manufactured by the following possible manufacturing process. A Littleford® FM130 granulator is charged with approximately one half of the guaifenesin, USP required for the process unit (about 17.4 kg) followed by about 4.00 kg of Methocel®, USP E10M Premium CR Grade; and 1.20 kg of Povidine, USP; and the balance of the guaifenesin (about 17.40 kg). The powder bed is dry mixed in the Littleford® FM130 granulator, with choppers on, for approximately 1 minute. At the completion of the 1-minute pre-mix cycle, about 12.0±0.05 kg of Purified Water, USP are sprayed onto the powder bed at a rate of about 2.40±0.24 kg/minute. Immediately following the addition of the Purified Water, USP, the unit is granulated for about 5 minutes. Hydrocodone granules are processed in a similar fashion. The granulated unit is discharged into double polyethylene-lined containers and then manually loaded into a Glatt® bowl while being passed through a #4 mesh screen. The Glatt® bowl is loaded into a Glatt TFO-60 fluid-bed drier with an inlet air temperature setting of about 70° C.±0.5° C. The unit is dried until a moisture level of 0.1±1.0% is obtained as determined using a Computrac® Moisture Analyzer, model MASA. The dried granulation is discharged into appropriately labeled, double polyethylene-lined drums and reconciled. The dried and reconciled granulation is then passed through a Kemutec® BetaGrind mill equipped with a 1.5 mm screen and running at approximately 1500 RPM. The milled granulation is collected into appropriately labeled, double polyethylene-lined drums and reconciled. The milled granulation is sampled and tested by Quality Control and released prior to further processing. The released guaifenesin and hydrocodone granulation units are charged to a Patterson-Kelley 20 ft3 V-blender after which they are blended together for about 10±1 minutes and then discharged to appropriately labeled, double polyethylene-lined containers.

Guaifenesin and hydrocodone tablets are formulated from a common granulation which is blended with appropriate quantities of Methocel®, USP E10M Premium CR Grade and Stearic Acid, NF to achieve the final dosage formulation.

### Formulation 2:

Dense pellets of guaifenesin and hydrocodone are prepared according to the wet granulation procedure for Formulation 1 as described in Example 7. Sized granules of guaifenesin are coated with an aqueous ethylcellulose dispersion (SURERELEASE™) in a fluid bed with Wurster configuration, using a temperature of 42-44° C. and a spray rate of 0.5 g/min. Delayed release granules of hydrocodone are coated with a latex dispersion of acrylic acid polymers (Eudragit® RS) in a fluid bed with Wurster configuration. Guaifenesin and hydrocodone tablets are manufactured as in Formulation 1.

Modifications and variations of the present invention will be apparent to those skilled in the art in view of the foregoing description and are intended to be encompassed by the following claims. All references cited herein are specifically incorporated by reference.

We claim:

1. A mixed release composition for oral administration comprising two or more drugs acting on different symptoms formulated for a combination of immediate release, extended release, and delayed release, wherein the first drug is sequentially released prior to release of the second or more drugs, to provide an effect that enhances action of the second drug.

2. The composition of claim 1, wherein the two or more drugs are selected from the group consisting of decongestants, antihistamines, expectorants, antitussives, cough suppressants, drying agents, and combinations thereof.

3. The composition of claim 2, wherein the decongestant is selected from the group consisting of phenylephrine, phenylpropanolamine, pseudoephedrine, pharmaceutically acceptable salts thereof, and combinations thereof.

4. The composition of claim 2, wherein the antihistamine is selected from the group consisting of chlorpheniramine, brompheniramine, dexchloropheniramine, dexbromopheniramine, triprolidine, diphenhydramine, doxylamine, tripelennamine, cyproheptatine, bromodiphenhydramine, phenindamine, pyrilamine, azatadine, pharmaceutically acceptable salts thereof and combinations thereof.

5. The composition of claim 2, wherein the expectorant is selected from the group consisting of guaifenesin, terpin hydrate, (glyceryl guaiacolate),, potassium guaiacolsulfonate, pharmaceutically acceptable salts thereof, and combinations thereof.

6. The composition of claim 2, wherein the antitussive is selected from the group consisting of caramiphen, dextromethorphan, codeine, hydrocodone, pharmaceutically acceptable salts thereof, and combinations thereof.

7. The composition of claim 2, wherein drying agent is selected from the group consisting of methscopolamine, scopolamine, meclizine, trimethobenzamide, dimenhydrinate, cyclizine, pheniramine, buclizine, chlorpheniramine, diphenhydramine, homatropine, carbinoxamine, clemastine, brompheniramine, and doxylamine.

8. The composition of claim 2, wherein the cough suppressant is selected from the group consisting of benzonatate, bromodiphenhydramine, brompheniramine, buclizine, carbinoxamine, chlorpheniramine, clemastine, codeine, cyclizine, dexchloropheniramine, dextromethorphan, dihydrocodeine, dimenhydrinate, diphenhydramine, doxylamine, homatropine, hydrocodone, meclizine, pheniramine, phenyltoloxamine, pyrilamine, trimethobenzamide, triprolidine.

9. The composition of claim 2 comprising an expectorant of immediate and extended release, wherein over 90% of the drug is released substantially linearly over a 12 hour period of time, and a cough suppressant for delayed and extended release, wherein over 90% of the drug is released between 4 and 12 hours, wherein the release profiles of the ingredients are orchestrated to coordinate the production and clearance of mucus and cough suppression.

10. The composition of claim 2 comprising:
(a) an anti-tussive active drug within a granule matrix packaged for extended release of over 90% of the active drug released substantially linearly over a 12 hour period of time;
(b) an anti-histamine active drug packaged for extended release of over 90% of the active drug released substantially linearly over a 12 hour period of time; and
(c) a drying agent active drug packaged for delayed and extended release of over 90% of the active drug released substantially linearly over a period of 8 hours following an initial delay of release of from about two to about four hours.

11. A composition of claim 2 wherein the pharmaceutical composition comprises:
(a) an immediate release decongestant active drug packaged for immediate release of over 60-80% of the active drug within from about 90 minutes to about 120 minutes following dosing;
(b) an extended release decongestant active drug packaged for extended release of over 90% of the active drug released substantially linearly over a 12 hour period of time; and
(c) a drying agent active drug packaged for delayed and extended release of over 90% of the active drug released substantially linearly over an 8 hour period following an initial delay of release of from about two to about 4 hours.

12. The composition of claim 9 wherein the pharmaceutical composition includes:
(a) an immediate release expectorant active drug packaged for immediate release of over 60-80% of the active drug within from about 90 minutes to about 120 minutes following dosing;
(b) an extended release expectorant active drug packaged for extended release of over 90% of the active drug released substantially linearly over up to at least a 12 hour period of time;
(c) an antitussive active drug packaged for delayed and extended release of over 90% of the antitussive active drug released substantially linearly over up to at least an 8 hour period following an initial delay of release of from about 2 to about 4 hours; and
(d) optionally a drying agent active drug packaged for delayed and extended release of over 90% of the active drug released substantially linearly over up to at least an 8 hour period following an initial delay of release of from about 2 to about 4 hours.

13. The composition of claim 9 wherein the pharmaceutical composition includes:
(a) an immediate release expectorant active drug packaged for immediate release of over 60-80% of the active drug within from about 90 minutes to about 120 minutes following dosing;
(b) an extended release expectorant active drug packaged for extended release of over 90% of the active drug released substantially linearly over up to at least a 12 hour period of time;
(c) an antitussive active drug packaged for delayed and extended release of over 90% of the active drug released substantially linearly over up to at least an 8 hour period following an initial delay of release of from about 2 to about 4 hours; and
(d) optionally a drying agent active drug packaged for delayed and extended release of over 90% of the active drug released substantially linearly over up to at least an 8 hour period following an initial delay of release of from about 2 to about 4 hours.

14. The composition of claim 9 wherein the pharmaceutical composition includes:
(a) an extended release expectorant active drug packaged for extended release of over 90% of the expectorant active drug released substantially linearly over up to at least a 12 hour period of time; and
(b) an antitussive active drug formulated for delayed and extended release, wherein over 90% of the antitussive active drug is released substantially linearly over up to at least an 8 hour period following an initial delay of release of from about 2 to about 4 hours.

15. The composition of claim 9 wherein the pharmaceutical composition includes:
(a) an extended release expectorant active drug packaged for extended release of over 90% of the expectorant active drug released substantially linearly over up to at least a 12 hour period of time; and
(b) an antitussive active drug packaged for sustained release such that up to 20% of the antitussive active drug can be released in a sustained fashion over the first 1 to 4 hours, followed by the sustained release of up to 100% of the antitussive active drug between 4 to 12 hours.

16. The composition of claim 9 wherein the pharmaceutical composition includes:
(a) an extended release expectorant active drug packaged for extended release of over 90% of the expectorant active drug released substantially linearly over up to at least a 12 hour period of time; and
(b) an antitussive active drug packaged for sustained release wherein the antitussive active drug is released in a first immediate release pulse, such that up to 50% of the antitussive active drug is released in a sustained fashion over the first 1 to 4 hours, followed by a sequential immediate release pulse of the antitussive active drug, such that up to 100% of the antitussive active drug is released between 4 to 6 hours.

17. The composition of claim 9 wherein the pharmaceutical composition includes:
(a) an extended release expectorant active drug packaged for extended release of over 90% of the expectorant active drug released substantially linearly over up to at least a 12 hour period of time; and
(b) an antitussive active drug packaged for immediate and sustained release, such that up to 50% of the antitussive active drug is released in immediate fashion over the first 1 to 4 hours, followed by sustained release of the antitussive drug, such that up to 100% of the antitussive active drug is released between 4 to 12 hours.

18. The composition of claim 9 wherein the pharmaceutical composition includes:
(a) an extended release expectorant active drug packaged for extended release,
wherein 20 to 35% of the expectorant active drug is released between 0 to 20 hours, 35-65% of the expectorant active drug is released between 2 to 4 hours, and 65-90% of the expectorant active drug is released between 4 to 8 hours; and
an antitussive active drug.

19. The composition of claim 9, wherein the pharmaceutical formulation is in a form selected from the group consisting of a capsule, tablet, oral rapid dissolving tablet, softgel, gelcap, film, gum, pastille, lozenge, disk, and wafer.

20. The composition of claim 9 wherein the anti-tussive active drug is dextromethorphan in the form selected from the group consisting of an immediate release bead, an immediate release matrix, a sustained release bead, a sustained release matrix, and combinations thereof; the anti-histamine active drug is chlorpheniramine in a form selected from the group consisting of an immediate release head, an immediate release matrix, a sustained release bead, a sustained release matrix, and combinations thereof; and the drying agent active drug is methscopolamine in a form selected from the group consisting of an immediate release bead, an immediate release matrix, a sustained release bead, a sustained release matrix, and combinations thereof.

21. The composition of claim 9 wherein the decongestant is pseudoephedrine in the form selected from the group consisting of an immediate release bead, an immediate release matrix, a sustained release bead, a sustained release matrix, and combinations thereof.

22. The composition of claim 9 wherein the expectorant is guaifenesin in the form selected from the group consisting of an immediate release bead, an immediate release matrix, a sustained release bead, a sustained release matrix, and combinations thereof.

23. The composition of claim 9 wherein the antitussive is hydrocodone in a form selected from the group consisting of an immediate release bead, an immediate release matrix, a sustained release bead, a sustained release matrix, and combinations thereof.

24. The composition of claim 9 wherein the tablet is in the form of a rapid dissolving tablet.

25. The composition of claim 9, comprising different types of coated beads or granules into a capsule, wherein each type of coated bead or granule includes individually an extended release anti-tussive, an extended release antihistamine and drying agent in a delayed release form, and wherein the capsule further includes one or more excipients.

26. The composition of claim 9, comprising a mixture of excipients and a mixture of granules into a tablet, wherein each type of granule includes individually an extended release anti-tussive, an extended release antihistamine and a drying agent in a delayed release form.

27. The composition of claim 9 wherein the mixture of excipients further includes free active drug.

28. The composition of claim 9 comprising a formulation of hydrocodone and guaifenesin, wherein release of hydrocodone is achieved in a series of sequential immediate release pulses for up to 12 hours after administration.

29. The composition of claim 9 comprising hydrocodone and guaifenesin in claim 9, wherein 25-50% of the hydrocodone is released as an immediate release pulse within 1-4 h after administration, and 50-100% of the hydrocodone is released as a second immediate release pulse within 4 to 12 hours after administration.

30. The composition of claim 9 comprising hydrocodone and guaifenesin in claim 9, wherein up to 100% of the hydrocodone is released as a single immediate release component 2 to 15 hours after administration.

31. The composition of claim 9, comprising hydrocodone and guaifenesin, wherein 25-50% of the hydrocodone is released in a sustained fashion during the first 1 to 4 hours after administration, which is followed by sustained release of 25 to 100% of the hydrocodone between 4 to 12 hours after administration.

32. The composition of claim 9 comprising hydrocodone and guaifenesin, wherein (i) 20 to 35% of guaifenesin is released within 2 hours of administration, (ii) 35-65% is released between 2 to 4 hours after administration and (iii) 65-90% is released between 4 to 8 hours after administration.
